(19) United States
(12) Patent Application Publication
(10) Pub. No.: US 2007/0184559 A1
(43) Pub. Date: Aug. 9, 2007

(54) Method and compounds for the fluorescent labelling of biomolecules and polymer particles

(76) Inventors: Sergey M. Yarmoluk, Kiev (UA); Olexandr M. Kostenko, Kiev (UA); Oleksiy Iwanowitch Tolmachev, Kiev (UA); Otto S. Wolfbeis, Rogensburg (DE)

Correspondence Address: FULBRIGHT & JAWORSKI, LLP, 666 FIFTH AVE, NEW YORK, NY, 10103-3198, US

(21) Appl. No.: 11/639,544

(22) Filed: Dec. 15, 2006

Related U.S. Application Data
(63) Continuation of application No. 10/287,450, filed on Nov. 4, 2002, now abandoned.

(30) Foreign Application Priority Data
Nov. 5, 2001 (DE) 101 53 818.9

(51) Int. Cl. G01N 33/543; G01N 1/30; C07D 405/02

(52) U.S. Cl. 436/518; 435/40.5; 546/282.1

(57) ABSTRACT

Hence the invention concerns a method which can be used to provide a fluorescent label on bioorganic molecules carrying amino groups such as amino acids, proteins, pharmaceutical agents, antibodies, amino group-modified nucleotides and also polymers and polymer particles carrying amino groups by means of a chemical (covalent) binding. The method is based on the reaction of a pyrylium salt located on a fluorophore F with the amino group of a biomolecule or particle according to the described reaction equation. The method is selective, simple to carry out and results in high labelling yields. The spectral properties of the conjugates differ considerably from those of the starting compounds and their fluorescence quantum yields are often considerably increased.

The invention concerns a method for the fluorescent labelling of substances carrying amino groups, fluorescent labels that are suitable for this method and their application in fluorescence-based analytical or diagnostic methods of determination.

The fluorescent labelling of biomolecules plays an important role in bioanalytics and biological research. A distinction is made between fluorophores which bind non-covalently to biomolecules such as proteins or DNA and those which can be bound covalently to biomolecules and also to particles.

The general reaction scheme for all known methods can be described as follows: A group X is located on a fluorophore F and can chemically react with a second group (e.g. HY) located on a biomolecule or particle (or alternatively only strongly interacts as is the case for example between biotin and avidin). If chemical (covalent) bonds are formed, either a group of the type XH is cleaved off in this process typically according to the following reaction equation:
F—X+HY biomolecule==>F-biomolecule+XH

However, the conjugation can also be like an addition reaction according to the following equation:
F—X+HY biomolecule==>F—XH—Y biomolecule

In this manner a fluorophore F can be introduced into a molecule thus making it detectable by all analytical methods based on fluorescence. Typical examples of groups X and Y are given in Table 1 for (a) substitution, (b) addition and (c) binding reactions:

| | Y (on the | |
|---|---|---|
| X (on the | biomolecule | Reaction type |
| fluorophore) | or particle) | (leaving group or new group) |
| —SO₂Cl | R′—NH₂ | substitution (—HCl) |
| —CO—CH₂—I | R′—SH | substitution (-Hl) |
| —CO—CH₂—Br | R′—COOH | substitution (—HBr) |
| —CO—O—NHS*⁾ | R′—NH₂ | substitution (-N-hydroxy- |
| | | succinimide) |
| —NCS | R′—NH₂ | addition (—NH—CS—NH—R′) |
| —NCO | R′—OH | addition (—NH—CO—OR′) |
| | (alcohol) | |
| -maleinimide | R′—SH | addition |
| -biotin | R′-avidin | affinity binding (non-covalent) |
| -avidin | R′-biotin | affinity binding (non-covalent) |

*⁾NHS = N-hydroxysuccinimide

However, it is often difficult to introduce the reactive groups (X) by chemical synthesis and they also have the disadvantage that they are not stable on storage. Even traces of water can slowly decompose such groups (by hydrolysis) and they become unreactive. This applies in a similar manner to biotinylation.

Hence one object of the present invention was to provide a method for fluorescent labelling which does not have the said disadvantages. In particular the intention was to provide fluorescent labels which are stable towards hydrolysis and on storage.

This object is achieved according to the invention by a method for the fluorescent labelling of substances carrying amino groups which is characterized in that a fluorescent dye which contains at least one reactive pyrylium group of the following structures A, B or C 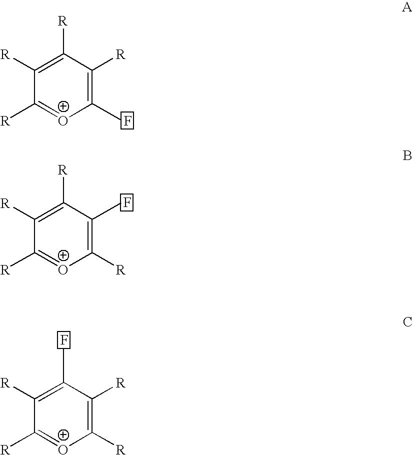
in which

F represents a fluorophore,

R represents a residue which does not quench the fluorescence of the fluorophore and does not hinder the reaction of the pyrylium group with amines,
is reacted with the primary amino group of the substance carrying amino groups to form a pyridinium salt of the structure D according to the reaction 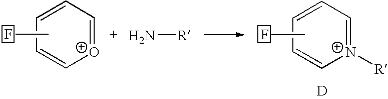
in which F represents a fluorophore and

R′ represents a (bio)organic residue.

The new method described here produces fluorophores with a reactive group (X) which can have one of the following chemical structures A, B or C: 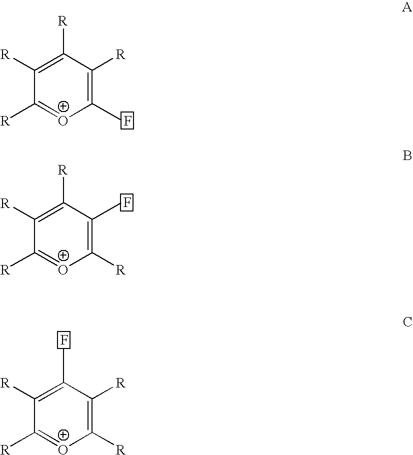
F represents any fluorophore and R represents any predominantly organic substituent which does not quench the fluorescence of the system and does not hinder the reaction of the pyrylium salt with amines. This type of reactive group is referred to herein as a pyrylium group.

It was found that such dyes can be used to fluorescently label species carrying amino groups. The following reaction occurs with primary amines (R′—NH₂) under relatively mild conditions in aqueous as well as in organic solvents: 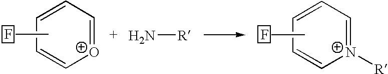
in which F again represents any fluorophore and R′ represents a (bio)organic and in particular an aliphatic or aromatic residue with for example 1 to 30 and in particular 1 to 20 C atoms which can also be substituted as desired. In this manner a biomolecule fluorescently labelled with F can be obtained from a non-labelled biomolecule having an amino group (R′—NH₂). By definition secondary amines (R′—NH—R′) and tertiary amines (NR′₃) are not labelled.

The method according to the invention can be generally used to label substances which contain at least one primary amino group.

Preferred primary amines are aliphatic and aromatic amines, amino acids and amino-modified biomolecules and pharmaceutical agents and also synthetic materials and polymers and polymer particles with free amino groups. The polymer particles preferably have a diameter between 0.1 and 20 μm and more preferably between 1 and 10 μm.

According to the invention the group F can be any fluorophore i.e. a residue which has fluorescent properties. The residues R on the pyrylium group are preferably hydrogen or hydrocarbon residues with 1 to 30 C atoms, preferably 1 to 10 C atoms. Examples of particularly preferred residues R are hydrogen, methyl, tert.butyl and phenyl. Pyrylium compounds are particularly preferred in which the group F is located in the para-position i.e. compounds of structure C. In addition it is preferred that the residue R in position 2 and 6 (ortho) is different from hydrogen.

The fluorescent labels according to the invention are particularly suitable for labelling substances carrying amino groups for fluorescence-based analytical or diagnostic methods of determination. A simple optical detection of the analyte is possible by covalently binding the analyte i.e. a substance carrying amino groups, to the fluorescent label according to the invention. A special characteristic of the fluorescent labels according to the invention is that the pyrylium group can undergo a covalent chemical reaction with primary amino groups which results in a covalent fluorescent labelling of substances which contain an amino group. Hence the method described here concerns labels that can bind covalently to biomolecules (and particles) containing amino groups.

Hence the invention concerns in particular a method which can be used to provide fluorescent labels via a chemical (covalent) binding on bioorganic molecules carrying amino groups such as amino acids, proteins, pharmaceutical agents, antibodies, nucleotides modified with amino groups and also polymers and polymer particles carrying amino groups. The method is based on the reaction of a pyrylium salt on a fluorophore F with the amino group of a biomolecule or particle according to the aforementioned reaction equation. The method is selective, simple to carry out and results in high labelling yields. The spectral properties of the conjugates differ considerably from those of the starting compounds and they often have considerably increased fluorescence quantum yields.

The reaction is elucidated by the following typical examples.

### EXAMPLE 1

### Synthesis of the Marker Dye Cyan 39: (1-methyl-2-[4-(2,6-dimethyl-1,4-dihydropyrylidene)methyl]-benzo-1,3-thiazolium-perchlorate

5.5 g 1,2-dimethyl-benzo-1,3-thiazolium methosulfonate and 2.48 g 2,6-dimethyl-4-pyrone are dissolved with 1 drop of perchloric acid in 10 ml acetic anhydride and refluxed for 4 h. Afterwards the reaction mixture is diluted with 20 ml ethanol and 2 ml of a saturated aqueous sodium perchlorate solution is added. After 2-3 h the resulting yellow precipitate is suction filtered and crystallized from ethanol.

### EXAMPLE 2

### Synthesis of the Marker Dye Cyan 58: 2,6-dimethyl-4-[3-(1-methyl-2,3-dihydro-1,3-benzthiazol-2-ylydene)-1-propenyl]-pyrylium-perchlorate

A mixture of 150 mg 2,4,6-trimethylpyrylium tetrafluoroborate and 218 mg 2-[2-(acetanilinovinyl-1)]-3-methylbenzthiazolium iodide is boiled for 1 h with 50 mg triethylamine in 5 ml 100% ethanol. Afterwards 500 μl of a saturated aqueous solution of sodium perchlorate is added. After cooling the resulting blue-violet precipitate is removed by filtration and purified by chromatography on aluminium oxide (mobile solvent: dichloromethane). The blue-violet fraction is isolated and, after evaporation of the mobile solvent, is obtained in a pure form by crystallization from ethanol.

Tables 2-4 show further chemical structures of dyes according to the invention, the absorption maxima of the free pyrylium salts (left column) and their conjugates with the amino acid glycine (right column). The absorption maxima of the conjugates having the longest wavelengths are usually shorter than those of the non-conjugated pyrylium dyes. The spectra are almost unaffected by the type of amine or amino acid. However, the table shows that the colour of the dyes can be adjusted as desired by varying the substituent F on the pyrylium salt.

TABLE 2
Chemical structures of pyrylium salts (left) and
their reaction products with glycine (R = CH₂—COOH) (right)

| | | λₘₐₓ, |
|---|---|---|
| No. | chem. Structure | nm |
| 1 | 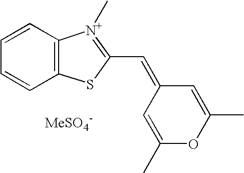 | 432, 460 |
| 2 | 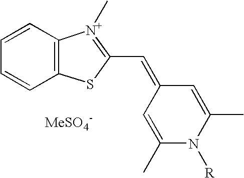 | 440 |
| 3 | 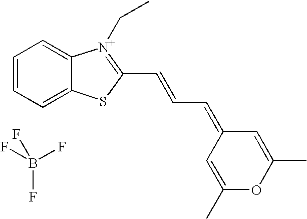 | 536 572 |
| 4 | 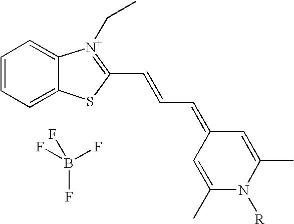 | 540 |
| 5 | 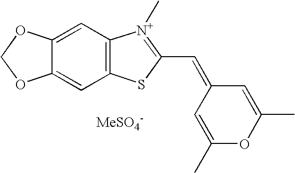 | 461 480 |
| 6 | 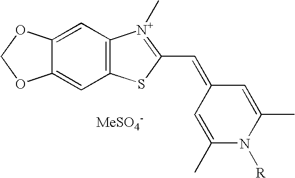 | 460 |
| 7 | 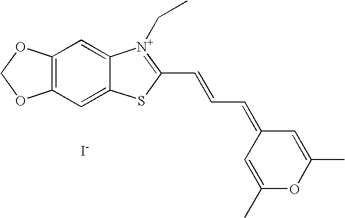 | 558 592 |
| 8 | 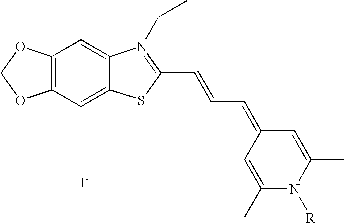 | 567 |

TABLE 3
Further examples of pyrylium salts (left) and
their reaction products with glycine (R = CH₂—COOH) (right)

| | | λₘₐₓ, |
|---|---|---|
| No. | chem. Structure | nm |
| 9 | 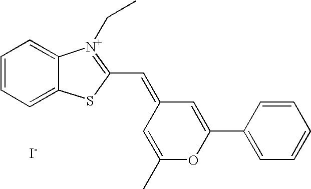 | 460 483 |
| 10 | 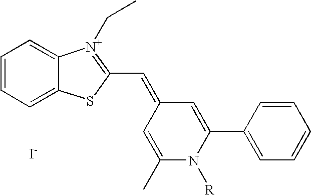 | 450 |
| 11 | 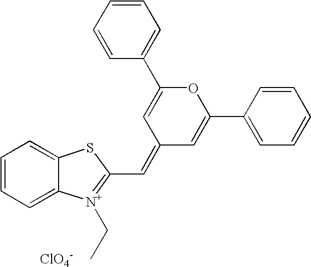 | 476, 502 |
| 12 | 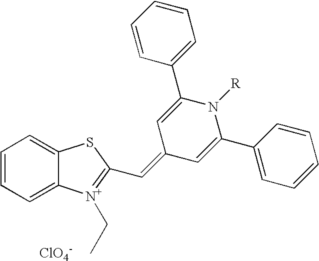 | 462 |
| 13 | 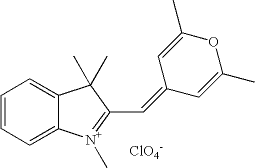 | 453 |
| 14 | 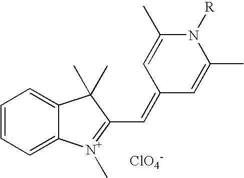 | 432 |
| 15 | 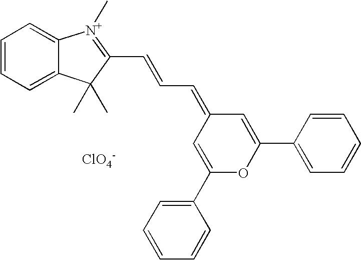 | 589, 628 |
| 16 | 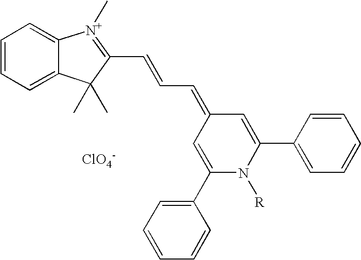 | 550 |
| 17 | 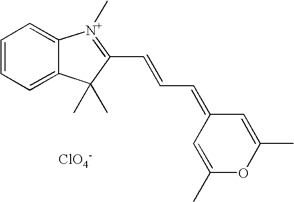 | 554, 575 |
| 18 | 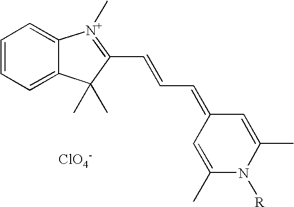 | 509 |
| 19 | 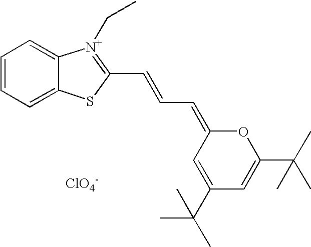 | 587, 626 |
| 20 | 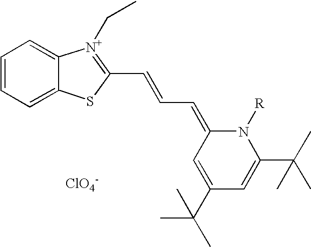 | 547 |

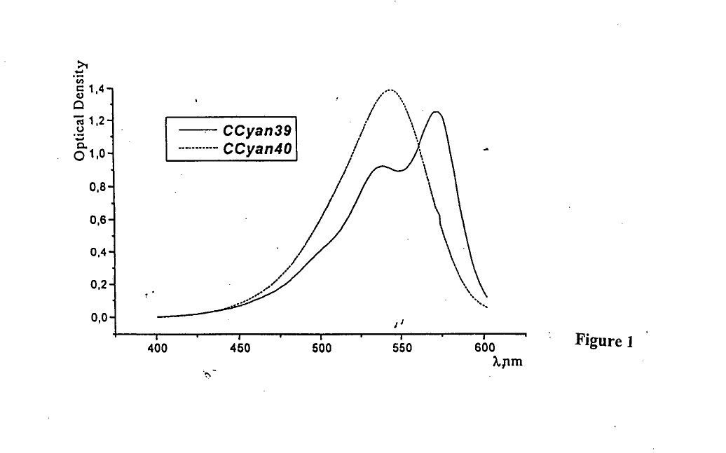

The absorption maxima of the pyrylium dyes (left column in tables 2-4) usually have two absorption bands i.e. a more intensive band in the longer wavelength region and a weaker band in the shorter wavelength region. In contrast the conjugates with the amine usually have a single strong band which is usually near to the shorter wave band of the pyrylium salt. The absorption spectrum of the compound of example 2 is shown in FIG. 1. The continuous line of the blue-violet label CCyan 39 is the typical 2-band spectrum of a pyrylium salt before conjugation to an amino group. The dotted line is the typical one-band spectrum of the dye after conjugation of the amino acid glycine (curve CCyan 40).

### EXAMPLE 3

### Conjugation to Amino Acids

The labels described above were used to fluorescently label amino acids. TEAA buffer (triethylammonium acetate) and dimethylsulfoxide were used as solvents. The respective amino acid was dissolved in alkaline buffer (<10) at a concentration of 0.1 mol/l. The dye Cyan 39 was dissolved in 2 ml DMSO (0.01 mol/l) and slowly added dropwise to the solution of the amino acid heated to 50° C. The reaction was monitored by the decrease in the light absorbance at 470 nm. Cyan 39 has an absorption maximum at 470 nm, the conjugate has its maximum at 434 nm. Cyan 58 can be used in a completely analogous manner. The conjugates can be purified by preparative chromatography (as described above).

The yellow- or red-labelled amino acids are formed particularly rapidly when dissolution is carried out at pH values above 10. The following table 4 gives an overview of the reaction times as a function of the adjusted pH. This shows that the reaction with the amino acid lysine (which is preferably labelled in proteins) is complete within 15-20 min at pH values between 11 and 12.

TABLE 4
Duration of the reaction (in minutes) until amino acids have been converted by
at least 90% with the label Cyan 39 in aqueous TEAA buffer at various pH values

| pH | | | | | | | | | | |
|---|---|---|---|---|---|---|---|---|---|---|
| value | ε-ACS | Lys | Ser | Arg | Gly | Ala | Val | Trp | His | Asp |
| 12.0 | 11 | 13 | 30 | 38 | 8 | 47 | 51 | 56 | 70 | 66 |
| 11.4 | 18 | 20 | 35 | 51 | 16 | 66 | 78 | 106 | 113 | 109 |
| 11.2 | 33 | 26 | 62 | 166 | 22 | 85 | 104 | 130 | 145 | 185 |

### EXAMPLE 4

### Labelling an Amino-Modified Oligomer

The amino-modified 15-oligomer 3′-TAA TGG CCT GAG ATAT-(CH₂)₆—NH₂ was reacted with the reactive dye Cyan 58 in the following manner: The oligomer (0.2 mg) was dissolved in 5 ml acetonitrile and heated to 50° C. Then a solution of 0.1 mg of the dye Cyan 58 was slowly added dropwise. After 1 h the acetonitrile was removed by evaporation and the residue was subjected to a polyacrylamide electrophoresis. The oligomer and remaining (excess) dye can be easily separated. The free label is blue-violet and has a maximum absorption at 572 nm. The conjugate with the oligomer is red-violet and has a maximum absorption at 540 nm.

### EXAMPLE 5

### Labelling of Glass Microparticles

Porous glass beads (1.0 g; pore size 70 nm) with aminopropyl groups on the surface (40-100 μmol per gram beads; obtained from Sigma, prod. No. G-5019) were suspended in a buffer solution of pH 10. A solution of 3 mg of the blue dye Cyan 58 (see above) in DMSO was slowly added dropwise while stirring rapidly at 50° C. After one hour the violet stained glass particles were suction filtered, washed with copious amounts of distilled water, 1% acetic acid and again with water until dye was no longer detected in the wash water. Afterwards the particles were dried and stored in a dried state. The violet coloured particles have a strong orange-red fluorescence.

### EXAMPLE 6

### Labelling of Polystyrene Microparticles

In order to demonstrate a fluorescence labelling of polystyrene particles, particles having an average diameter of 160-200 μm were used which carried aminomethyl groups on the surface (product 81558, from Fluka, Buchs; Switzerland). 1.0 g of these particles was suspended in ethanol, excess dye Cyan 39 (10 mg; see example 1) was added and refluxed for 2 h. Particles labelled in this manner are stained orange-red and have a green fluorescence.

### EXAMPLE 7

### Labelling of a Protein

100 μl of a 0.1 M solution of triethylammonium acetate buffer (of pH 12.1) was added to 100 μl of a solution of lysozyme (Sigma) in water (2 mg/ml, ≈1.5·10⁻⁴ M). Afterwards 100 μl of a solution (1.5·10⁻³ M) of the dye Cyan 39 in DMSO was added. The reaction mixture was kept for 2 h at 50° C. Subsequently the solution was applied to a Sephadex column (Sephadex G-25, column: 1 cm×30 cm, 0.01 M TEAA buffer, pH 12.1) and the orange-red free dye was separated in this manner from the yellow lysozyme conjugate. The yellow protein fraction runs much more rapidly than the dye.

1. A method for fluorescent labelling of a substance carrying a primary amino group, H2N—R′, wherein R′ is a protein residue, comprising reacting a fluorescent dye which contains at least one reactive pyrylium group of formula: 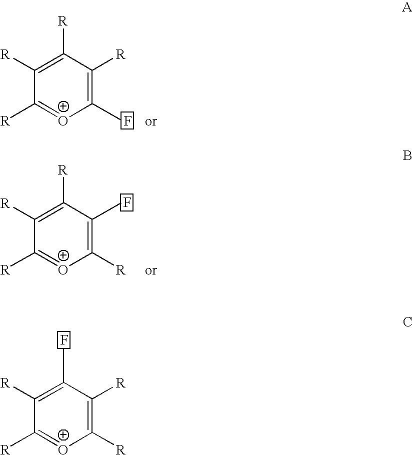
wherein
F represents a fluorophore linked to the pyrylium group through a—(CH═CH)ₙ linker, wherein n is 1 or 2,
R represents a residue which does not quench the fluorescence of the fluorophore and does not hinder reaction of the pyrylium group with amines, and is a hydrogen or a hydrocarbon residue with 1-30 C-atoms,
with the primary amino group of said substance carrying a primary amino group, to form a pyridinium salt of formula: [chemical structure]


SEQUENCE LISTING (1 sequence)

SEQ ID NO: 1 - DNA, 16 bp, Artificial sequence
  Portion of an oligomer reactive with Cyan 58
taatggcctgagatat